(19) United States
(12) Patent Application Publication
(10) Pub. No.: US 2021/0045773 A1
(43) Pub. Date: Feb. 18, 2021

(54) TRANSSEPTAL SHEATH WITH ANCHORING COIL FOR CONTROLLED LEFT ATRIAL ACCESS

(71) Applicant: Ethicon, Inc., Somerville, NJ (US)

(72) Inventors: Matthew Kuhn, Houston, TX (US); Jorge Salazar, Houston, TX (US); Christine Nicole Ballew, Southlake, TX (US); Rolando Marquez, Pasadena, TX (US); Ellen Marie McMullen, St. Petersburg, FL (US); William Patrick Porter, San Antonio, TX (US)

(21) Appl. No.: 16/937,693

(22) Filed: Jul. 24, 2020

Related U.S. Application Data
(60) Provisional application No. 62/886,411, filed on Aug. 14, 2019.

(51) Int. Cl. A61B 17/34; A61M 25/00; A61M 25/04; A61M 29/00

(52) U.S. Cl. CPC A61B 17/3468; A61M 25/0084; A61B 2090/3966; A61M 29/00; A61M 25/04

(57) ABSTRACT

A device may comprise a shaft comprising a hollow body, an anchor disposed adjacent an end of the shaft, wherein the anchor is configured to engage a surface to releasably secure the shaft to the surface, and a needle at least partially disposed within the shaft and is configured to be advanced toward the surface and outside of the shaft.

### CROSS-REFERENCE TO RELATED APPLICATIONS

This application claims the benefit of U.S. Provisional Patent Application No. 62/886,411 filed Aug. 14, 2019.

### BACKGROUND OF THE DISCLOSURE

### 1. Field of the Disclosure

The present disclosure relates to system(s), method(s), and device(s) for accessing the left atrium while preventing inadvertent needle puncture of cardiac structures and other complications.

### 2. Background of the Disclosure

Transseptal punctures are performed in various cardiac procedures that require access to the left side of the heart. An estimated 300,000 transseptal puncture procedures are performed annually in the United States. Due to its technically demanding nature, transseptal punctures require skilled operators and sound understanding of cardiac anatomy. Even with skilled physicians, transseptal procedures are accompanied by a high incidence of complications.

In some patients, the septum can be extremely compliant when tenting the fossa ovalis. In other patients, fibrotic scarring of the fossa and other abnormalities make transseptal punctures very difficult. In both cases, there is an increased risk of perforation of the heart wall following puncture of the fossa. There are no tools available that can adequately address this safety issue today.

Transseptal punctures (TSPs) are a commonly used procedure as a method of gaining access to the left atrium in a minimally invasive way. This procedure is used to facilitate various cardiovascular procedures, including but not limited to left atrial appendage closures, heart valve repairs, and cardiovascular device implantations. Due to a wide range of associated applications and medical procedures, around 300,000 transseptal punctures are performed per year in the United States.

One limitation of the procedure is that the target site for puncture, the fossa ovalis, accounts for only 20% of the area of the interatrial septum, making it difficult to locate. This difficulty is further exacerbated by the fact that the user must navigate and maneuver the catheter assembly to the interatrial septum without the aid of direct visualization.

A second limitation of the procedure is that controlled puncture of the fossa ovalis with a transseptal needle can be very difficult, especially for patients with an aneurysmal or fibrotic septum. About 40% of patients have an aneurysmal septum, meaning the septum (or the fossa ovalis) is more compliant (i.e. stretchy) and prone to distension during tenting with the dilator. As a result, an aneurysmal septum can allow for the sharp tip of a transseptal needle to come dangerously close to the heart wall of the left atrium before the needle finally punctures through the septum. Conversely, for patients with a fibrotic septum, the septum is less compliant (i.e. tougher) and less prone to distension during tenting with the dilator. As a result, a fibrotic septum can require the application of a significant amount of force to the transseptal needle before the needle is able to puncture through the septum. When puncture finally occurs, it is very difficult for the user to rapidly cease the application of force to the transseptal needle and—similarly to transseptal puncture of an aneurysmal septum—as a result, the user may unintentionally over-advance the transseptal needle and injury the heart wall leading to costly complications and even death.

Another drawback is that current visualization techniques for TSPs are limited in their ability to provide useful information to the user of a transseptal access device. Techniques such as fluoroscopy only allow for 2D projections of 3D space and therefore do not provide sufficient detail into device orientation and positioning in 3D space. Physicians performing TSPs often need several years of experience with the procedure before they are able to draw meaningful information from the visualization or the slight “drops” as the assembly is placed along the fossa ovalis.

Due to the challenges that arise with this procedure, TSPs are associated with 3,000 fatal complications, 51,000 repeat procedures, and 42,000 other issues each year out of the 300,000 performed annually in the United States. Therefore, there exists a need to address these issues and improve patient outcomes. Transseptal access devices are in need of a means to facilitate the process of locating the fossa ovalis. Transseptal access devices are also in need of a method to better control the advancement of transseptal needles through the septum before, during, and after puncture—especially in cases where a patient presents with an aneurysmal or fibrotic septum. Lastly, transseptal access devices are in need of a means to better determine device position and orientation within the body using conventional medical imaging technologies.

Thus, improvements are needed.

### 3. Discussion of the Related Art

To combat potential complications from arising during transseptal puncture procedures, these transseptal access devices often have sleek profiles, soft atraumatic distal tips, specific braid composite designs for ideal torque transmission, and/or hemostatic valves to prevent backflow and air ingress. Despite the sophistication of the market leader's devices, controlling the advancement of transseptal access devices into the left atrium during transseptal puncture remains a challenge, especially for patients with an atrial septum of atypical material properties (i.e. an aneurysmal and/or fibrotic septum). Furthermore, maintaining transseptal access for the introduction of additional devices into the left atrium also remains a challenge due to unintentional migration of transseptal access devices out of the left atrium after achieving access.

Accordingly, there exists a need for a reliable, cost effective system, method, and device for performing transseptal puncture in a safer, more controlled manner as well as a need for maintaining access to the left atrium after transseptal puncture has been achieved.

### SUMMARY OF THE DISCLOSURE

In the present disclosure, a device may comprise a shaft comprising a hollow body and an anchor disposed adjacent an end of the shaft, wherein the anchor is configured to engage a surface to releasably secure the shaft to the surface.

In the present disclosure, the device may additionally comprise a needle at least partially disposed within the shaft wherein the needle is configured to be advanced toward the surface and outside of the shaft.

In the present disclosure, a device may comprise a catheter comprising a hollow body configured to be disposed to enclose at least a portion of a transseptal sheath, wherein the device comprises a hollow body and an anchor disposed adjacent an end of the catheter, wherein the anchor is configured to engage a surface to releasably secure the catheter to the surface.

In the present disclosure, a device may comprise a catheter comprising a hollow body configured to be disposed to enclose at least a portion of a transseptal dilator, wherein the device comprises a hollow body and an anchor disposed adjacent an end of the catheter, wherein the anchor is configured to engage a surface to releasably secure the catheter to the surface.

In the present disclosure, a device may comprise a catheter comprising a hollow body configured to be disposed to enclose at least a portion of a transseptal needle, wherein the device comprises a hollow body and an anchor disposed adjacent an end of the catheter, wherein the anchor is configured to engage a surface to releasably secure the catheter to the surface.

In the present disclosure, a device may comprise a catheter comprising a hollow body configured to be disposed to enclose at least a portion of a transseptal access catheter assembly, wherein the device comprises a hollow body and an anchor disposed adjacent an end of the catheter, wherein the anchor is configured to engage a surface to releasably secure the catheter to the surface, and wherein the transseptal access catheter assembly comprises at least one of a transseptal sheath, a dilator, a transseptal sheath, and a guidewire.

In the present disclosure, a method may comprise disposing a device adjacent a biological surface, wherein the device comprises at least a shaft having a hollow body and a needle disposed at least partially within the hollow body of the shaft, causing the device to engage the surface to releasably secure at least a portion of the device to the surface, and causing the needle to be advanced toward the surface and outside of the hollow body of the shaft, while the at least a portion of the device is secured to the surface.

### BRIEF DESCRIPTION OF THE DRAWINGS

The foregoing and other features and advantages of the disclosure will be apparent from the following, more particular description of preferred embodiments of the disclosure, as illustrated in the accompanying drawings.

FIG. 1 is a diagrammatic representation of a human heart showing an example transseptal needle passing through the septum.

FIG. 2A is a diagrammatic representation of an example device in accordance with the present disclosure.

FIG. 2B is an exploded view of at least a portion of the components of the device shown in FIG. 2A.

FIG. 3A is a diagrammatic representation of an enlarged view of a portion of the device shown in FIG. 2A.

FIG. 3B is a diagrammatic representation of an enlarged view of a portion of the device shown in FIG. 2A, showing a needle extended.

FIG. 4A is a diagrammatic representation of an enlarged view of an example device in accordance with the present disclosure.

FIG. 4B is a diagrammatic representation of an enlarged view of a portion of the device shown in FIG. 4A, showing a needle extended.

FIG. 5A is a diagrammatic representation of an enlarged view of an example device in accordance with the present disclosure.

FIG. 5B is a diagrammatic representation of an enlarged view of a portion of the device shown in FIG. 5A, showing a needle extended.

FIGS. 6A-6G illustrate a diagrammatic representation of a sequence in accordance with an example method of the present disclosure.

FIG. 7 illustrates an example method flow in accordance with the present disclosure.

FIG. 8 illustrates an example conical stabilization element, where the dashed lines represent cavities or indentations within a cone into which spines may be disposed.

FIG. 9 illustrates an inner sheath configured to be coupled to the conical stabilization element of FIG. 8.

FIG. 10 is an exploded view of an overall assembly comprising the conical stabilization element of FIG. 8, the inner sheath of FIG. 9 and an outer sheath configured to receive at least a portion of the inner sheath.

FIG. 11 is an assembled view of the overall assembly comprising the conical stabilization element, the inner sheath and, and the outer sheath configured in an assembled configuration.

### DETAILED DESCRIPTION

The present relates to system(s), method(s) and device(s) for securing a biological surface (e.g., tissue) to allow a controlled puncture of the surface. The present disclosure may relate to system(s), method(s) and device(s) where access to the left atrium is needed. The present disclosure comprises a method and device configured for controlled transseptal puncture of the atrial septum. The present disclosure additionally comprises a method and device configured for maintaining access to the left atrium following transseptal puncture. The present disclosure comprises a catheter comprising a hollow body with an anchor disposed at a distal end of the catheter, wherein the anchor is configured to releasably secure the catheter to a biological surface such as the atrial septum or fossa ovalis. The catheter may additionally be configured to at least partially enclose at least one of a transseptal sheath, a dilator, a transseptal needle, and a guidewire.

The catheter of the present disclosure may be configured to at least partially enclose transseptal access catheter assemblies used in conventional transseptal puncture procedures, wherein the catheter is configured for the introduction of a transseptal sheath and wherein the catheter is slidably and rotationally disposed along the outer surface of the shaft of the transseptal sheath.

The catheter of the present disclosure may be configured to at least partially enclose a dilator used in conventional transseptal puncture procedures, wherein the catheter is configured for the introduction of a dilator and wherein the catheter is slidably and rotationally disposed along the outer surface of the shaft of the dilator.

The catheter of the present disclosure may be configured to at least partially enclose a dilator used in conventional transseptal puncture procedures, wherein the catheter is configured for the introduction of a dilator and wherein the catheter is slidably and rotationally disposed along the outer surface of the shaft of the dilator.

The catheter of the present disclosure may be configured to at least partially enclose a transseptal needle used in conventional transseptal puncture procedures, wherein the catheter is configured for the introduction of a transseptal needle and wherein the catheter is slidably and rotationally disposed along the outer surface of the shaft of the transseptal needle.

As an example, conventional transseptal puncture procedures as referenced herein may comprise use of catheter assemblies that may further comprise a transseptal needle that is housed within a dilator sheath. This dilator sheath is then housed within a final outer sheath. These three components may be configured to be slidably disposed relative to each other, and thereby the position of each component along their shared common axis can be adjusted by the user. As an illustrative example, a catheter assembly comprising a transseptal needle, a dilator, and a transseptal sheath may be used in the following manner to access the left atrium from the right atrium:
1. The catheter assembly is inserted into the femoral vein and guided through the inferior vena cava until the tip of the catheter reaches the superior vena cava.
2. The assembly is navigated into the right atrium of the heart.
3. The dilator is carefully advanced relative to the transseptal sheath until the tapered tip of the dilator protrudes from the distal/terminal end of the transseptal sheath.
4. The user approximates the tip of the dilator with the inner wall of the superior vena cave.
5. The user withdraws the assembly a distance until they feel a slight “drop” as the tip of the dilator moves from the superior vena cava to the interatrial septum of the right atrium.
6. The user withdraws the assembly further until they feel another slight “drop” as the assembly reaches the fossa ovalis, a region of the interatrial septum.
7. The tip of the dilator is positioned on the fossa ovalis and a gentle pressure is exerted onto the tissue by the user until the fossa ovalis tents inwards into the left atrium.
8. The user carefully advances the transseptal needle relative to the dilator while carefully retaining the position of the dilator and transseptal sheath relative to the atrial septum.
9. After the transseptal needle has punctured through the fossa ovalis into the left atrium, the dilator is slowly advanced through the fossa ovalis into the left atrium while the user carefully retains the position of the transseptal sheath relative to the atrial septum. The dilator functions to gently and gradually expand the puncture site through the fossa ovalis/atrial septum as performed by the transseptal needle.
10. Once the puncture site has been expanded by the dilator, the transseptal sheath is advanced along the dilator into the left atrium while the positions of the dilator and transseptal needle relative to the atrial septum are held steady to avoid accidental over-advancement into the left atrium,
11. After accessing the left atrium, the dilator and transseptal needle are removed from the transseptal sheath, which remains in the left atrium to allow for the introduction of additional devices through the transseptal sheath.

In an exemplary embodiment, the device of the present disclosure comprises a catheter with a helical coil disposed at the distal end of the catheter, wherein the helical coil of the device is configured to “anchor” the device to the atrial septum to provide structural support to the advancement of a transseptal needle during transseptal puncture. Furthermore, the anchoring action of the helical coil to the septum as described in the present disclosure allows for sustained maintenance of an access pathway into the left atrium for the introduction of other devices through the hollow body of the present disclosure.

The device may comprise an elongated tubular structure with a helical anchoring coil configured to be slidably and rotationally disposed along the shaft of at least one of a transseptal sheath, a dilator, a transseptal needle, and a guidewire. The device may additionally comprise a hollow body with a variable inner diameter that may function to limit the distal advancement of a transseptal needle through the hollow body of the device of the present disclosure. The device may comprise an elongated tube with an axis, a proximal end, a distal end, a hollow lumen extending longitudinally through, and depth position markings. The helical coil anchor of the device of the present disclosure may comprise a sharpened tip configured for the controlled advancement of the helical coil anchor into a target tissue site. The advancement of the helical coil anchor into a target tissue site may comprise rotating the device of the present disclosure in one direction to secure the catheter to the surface of the target tissue site. Conversely, releasing the helical coil anchor from a target tissue site may comprise rotating the device of the present disclosure in the opposite direction to disengage the helical coil anchor of the catheter from the target tissue site. Securing the device of the present disclosure to the atrial septum helps prevent excessive tenting of the atrial septum during transseptal puncture with a transseptal needle. Securing the device of the present disclosure to the atrial septum helps prevent unintentional perforation of the heart wall due to uncontrolled advancement of a transseptal needle through the atrial septum into the left atrium during transseptal puncture with a transseptal needle. Securing the device of the present disclosure to the atrial septum helps maintain access to the left atrium after transseptal puncture by helping prevent unintentional removal of a transseptal sheath from the left atrium.

Although shown and described in what is believed to be the most practical and preferred embodiments, it is apparent that departures from specific designs and methods described and shown will suggest themselves to those skilled in the art and may be used without departing from the spirit and scope of the disclosure. The present disclosure is not restricted to the particular constructions described and illustrated but should be constructed to cohere with all modifications that may fall within the scope of the appended claims.

The present disclosure comprises at least the following aspects:
Aspect 1. A device comprising:
an access sheath (e.g., catheter) comprising a hollow body configured to be disposed to enclose at least a portion of a transseptal sheath; and an anchor disposed adjacent an end of the access sheath, wherein the anchor is configured to engage a surface to releasably secure the access sheath to the surface.
Aspect 2. The device of aspect 1, wherein the hollow body of the access sheath is generally tubular.
Aspect 3. The device of any one of aspects 1-2, wherein the anchor comprises a coil.
Aspect 4. The device of any one of aspects 1-2, wherein the anchor comprises a coil configured to be advanced toward the surface in a first rotating motion to engage the surface and a second opposite rotating motion to disengage the surface.
Aspect 5. The device of any one of aspects 1-2, wherein the anchor comprises a tip or an edge configured to pierce at least a portion of the surface.
Aspect 6. The device of any one of aspects 1-5, wherein the surface comprises a biological surface.
Aspect 7. The device of any one of aspects 1-5, wherein the surface comprises a fossa ovalis of a heart.
Aspect 8. The device of any one of aspects 1-7, further comprising:
the transseptal sheath at least partially disposed within the hollow body of the access sheath; and
one or more of:
a transseptal needle at least partially disposed within the transseptal sheath; or
a dilator at least partially disposed within the transseptal sheath.
Aspect 9. The device of aspect 8, wherein the one or more of the transseptal needle or the dilator is configured to be advanced outside of the transseptal sheath.
Aspect 10. A device comprising:
a shaft comprising a hollow body;
an anchor disposed adjacent an end of the shaft, wherein the anchor is configured to engage a surface to releasably secure the shaft to the surface;
a needle at least partially disposed within the shaft and is configured to be advanced toward the surface and outside of the shaft.
Aspect 11. The device of aspect 10, wherein the hollow body of the shaft is generally tubular.
Aspect 12. The device of any one of aspects 10-11, wherein the anchor comprises a coil.
Aspect 13. The device of aspect 12, wherein the coil is disposed to encircle at least a portion of the shaft.
Aspect 14. The device of any one of aspects 10-11, wherein the anchor comprises a coil configured to be advanced toward the surface in a first rotating motion to engage the surface and a second opposite rotating motion to disengage the surface.
Aspect 15. The device of any one of aspects 10-11, wherein the anchor comprises a tip or an edge configured to pierce at least a portion of the surface.
Aspect 16. The device of any one of aspects 10-15, wherein the anchor is formed integrally with the end of the shaft.
Aspect 17. The device of any one of aspects 10-15, wherein the anchor is coupled to the end of the shaft.
Aspect 18. The device of any one of aspects 10-15, wherein the anchor is disposed along a longitudinal axis of the shaft and at least a portion of the anchor extends beyond the end of the shaft.
Aspect 19. The device of any one of aspects 10-18, wherein the surface comprises a biological surface.
Aspect 20. The device of any one of aspects 10-18, wherein the surface comprises a fossa ovalis of a heart.
Aspect 21. The device of any one of aspects 10-20, wherein the shaft comprises an access sheath and the device further comprises a transseptal sheath at least partially disposed within the hollow body of the access sheath, wherein the needle comprises a transseptal needle at least partially disposed within the transseptal sheath, and wherein the transseptal needle is configured to be advanced outside of the transseptal sheath.
Aspect 22. The device of aspect 21, further comprising a dilator at least partially disposed within the transseptal sheath.
Aspect 23. The device of aspect 22, wherein the dilator is configured to be advanced outside of the transseptal sheath.
Aspect 24. The device of any one of aspects 10-20, wherein the shaft comprises a catheter and the device further comprises a transseptal sheath at least partially disposed around the hollow body of the shaft, wherein the needle comprises a transseptal needle at least partially disposed within the transseptal sheath, and wherein the transseptal needle is configured to be advanced outside of the transseptal sheath.
Aspect 25. The device of aspect 24, further comprising a dilator at least partially disposed within the transseptal sheath.
Aspect 26. The device of aspect 25, wherein the dilator is configured to be advanced outside of the transseptal sheath.
Aspect 27. A method comprising:
disposing a device adjacent a biological surface, wherein the device comprises at least a shaft having a hollow body and a needle disposed at least partially within the hollow body of the shaft;
causing the device to engage the surface to releasably secure at least a portion of the device to the surface; and
causing the needle to be advanced toward the surface and outside of the hollow body of the shaft, while the at least a portion of the device is secured to the surface.
Aspect 28. The method of aspect 27, wherein the device comprises an anchor disposed adjacent an end of the shaft, wherein the anchor is configured to engage the surface to releasably secure the shaft to the surface.
Aspect 29. The method of aspect 28, wherein the anchor comprises a coil.
Aspect 30. The method of aspect 28, wherein the anchor comprises a coil configured to be advanced toward the surface in a first rotating motion to engage the surface and a second opposite rotating motion to disengage the surface.
Aspect 31. The method of aspect 28, wherein the anchor comprises a tip or an edge configured to pierce at least a portion of the surface.
Aspect 32. The method of aspect 27, wherein the hollow body of the shaft is generally tubular.
Aspect 33. The method of aspect 27, wherein the surface comprises a fossa ovalis of a heart.
Aspect 34. The method of aspect 33, further comprising causing the needle to be advance through at least a portion of an interatrial septum of the heart, while the at least a portion of the device is secured to the fossa ovalis.
Aspect 35. The method of aspect 34, further comprising causing the needle to be advance through at least a portion of the fossa ovalis of the heart, while the at least a portion of the device is secured to the fossa ovalis.
Aspect 36. The method of aspect 35, further comprising causing the needle to be advance into a left atrium of the heart, while the at least a portion of the device is secured to the fossa ovalis.

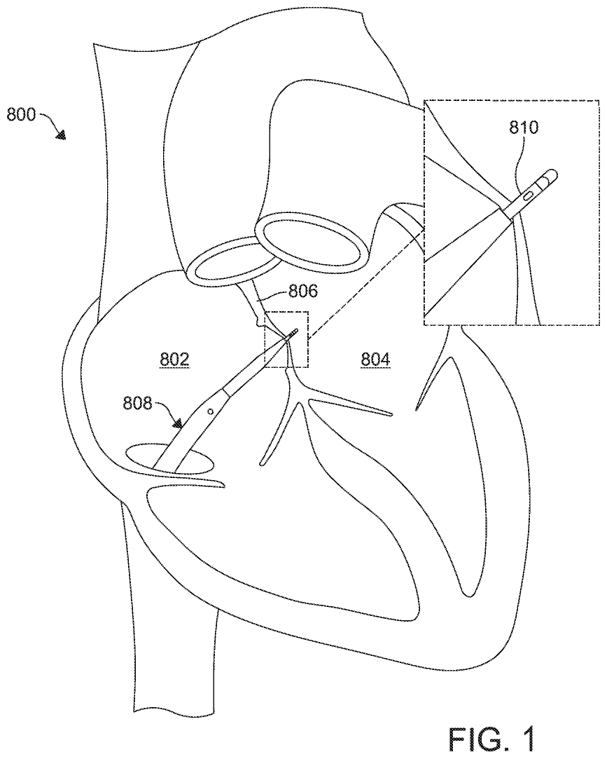

Commonly performed procedures to allow access to a left atrium for catheter ablation may include transseptal punctures. Historically, the conventional means used for the commonly performed procedures comprise mechanically puncturing the fossa ovalis, which may have serious complications, such as perforation of the heart wall, skiving of cardiovascular tissues, or puncture of the aorta. As an illustrative example, FIG. 1 shows a representation of a human heart 800 comprising right atrium 802 and a left atrium 804 separated by a septum 806. As an example, a device 808 may allow a transseptal needle 810 to puncture a portion of the septum 806 such as the fossa ovalis. However, current devices and needles may cause tenting in or around the puncture area.

Additionally, previous studies have evaluated the feasibility and safety of radiofrequency (RF) energy applied to a conventional needle as a technique to access a left atrium, particularly in patients with a repeat procedure, fibrotic septum, or aneurysmal septum. The use of RF energy to aid in transseptal punctures has increased in popularity, despite limited literature to support associated superiority and safety compared to the commonly performed procedures. Barriers to adoption still exist, particularly a limited availability of RF generators in catheterization laboratories.

A novel method and/or device is required to aid in the mechanical puncture of the fossa ovalis for access to the left atrium.

Disclosed herein is a device configured for releasably anchoring to at least a portion of the atrial septum to create a force-neutral mechanism to aid in an advancement of a transseptal needle into a left atrium.

Disclosed herein is a device comprising a high-torque catheter shaft outfitted with a helical anchoring coil at a most distal end. A lumen of said device is configured to enclose at least a portion of at least one of a transseptal sheath, a dilator, and transseptal needle, and a guidewire, and wherein the device is configured to be slidably and rotationally disposed along the shaft of a transseptal needle such that the transseptal needle may be advanced and retracted through the hollow body of the device for the purposes of puncturing through tissue. The needle may or may not comprise a hollow lumen.

A needle puncture into a fossa ovalis of a heart may create a small opening through which at least one of a guidewire, a dilator, and a transseptal sheath may be passed into a left atrium of the heart. A dilator may be advanced along the shaft of the transseptal need and/or along the shaft of a guidewire to widen the small opening created in the septum to allow for advancement of larger diameter devices into the left atrium, such as a transseptal sheath, an ablation catheter, a mapping catheter, a diagnostic catheter, an LVAD, and other devices.

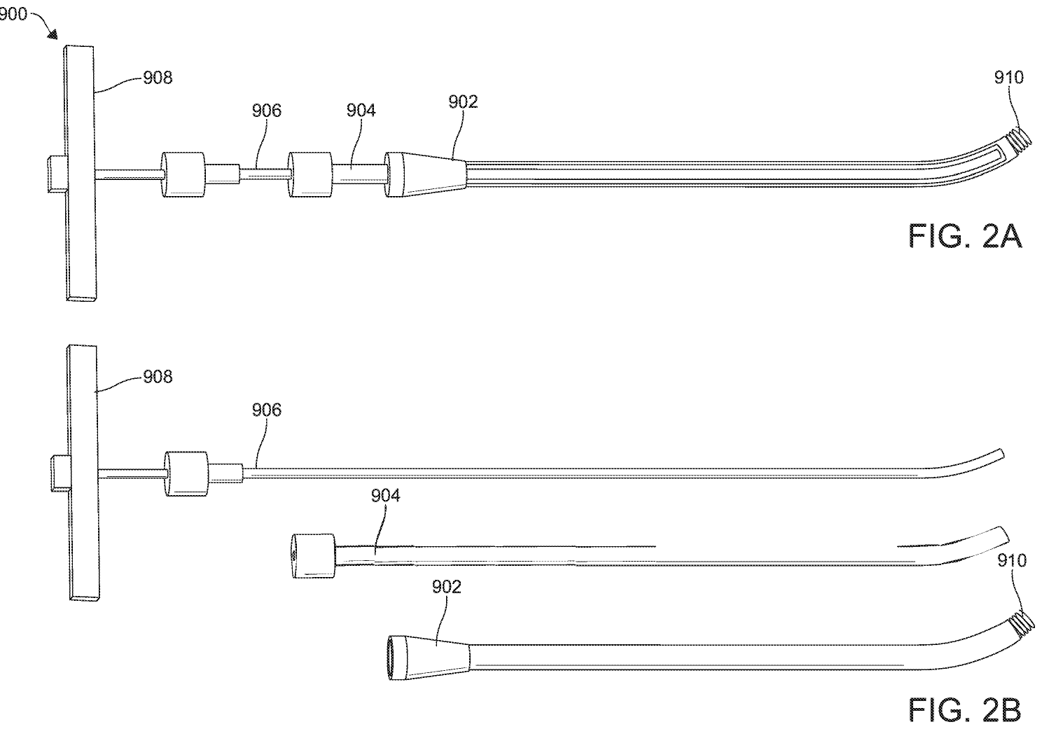

FIGS. 2A-2B illustrate an example device 900 comprising an outer sheath or access sheath 902, a transseptal sheath 904, a catheter 906, and a handle 908 configured to control a positioning and advancement of one or more of the components 902, 904, 906 or an article within the one or more components 902, 904, 906.

The access sheath 902 may comprise a hollow body. The hollow body may be sized and/or configured to be disposed to enclose at least a portion of another component such as a commercially available sheath or transseptal sheath 904, or other components. The hollow body of the access sheath 902 may be generally tubular. However other shapes and sizes may be used. An anchor 910 may be disposed adjacent an end of the access sheath 902, such as a distal end that is configured to be advanced. The anchor 910 may be configured to engage a surface to releasably secure the access sheath 902 or another component to the surface. The anchor 901 may comprise a coil, for example having a helical, corkscrew shape. The anchor 910 may comprises a coil configured to be advanced toward the surface in a first rotating motion to engage the surface and a second opposite rotating motion to disengage the surface. The anchor 910 may comprises a tip or an edge configured to pierce at least a portion of the surface. The surface may comprise a biological surface. The surface may comprise a septum or a fossa ovalis of a heart. Other surfaces may be used. Other configurations of the anchor 910 may be used.

The transseptal sheath 904 may comprise a commercially available transseptal sheath. The transseptal sheath 904 may be configured to be disposed within the hollow body of the access sheath 902. The transseptal sheath 904 may comprise a hollow body configured to receive one or more components therein and/or there through. One or more of a transseptal needle or a dilator may be at least partially disposed within the transseptal sheath and may be configured to be advanced outside of the transseptal sheath 904. As an example, the catheter 906 may be configured to be disposed within the transseptal sheath 904 and may be used to guide and control advancement of a transseptal needle.

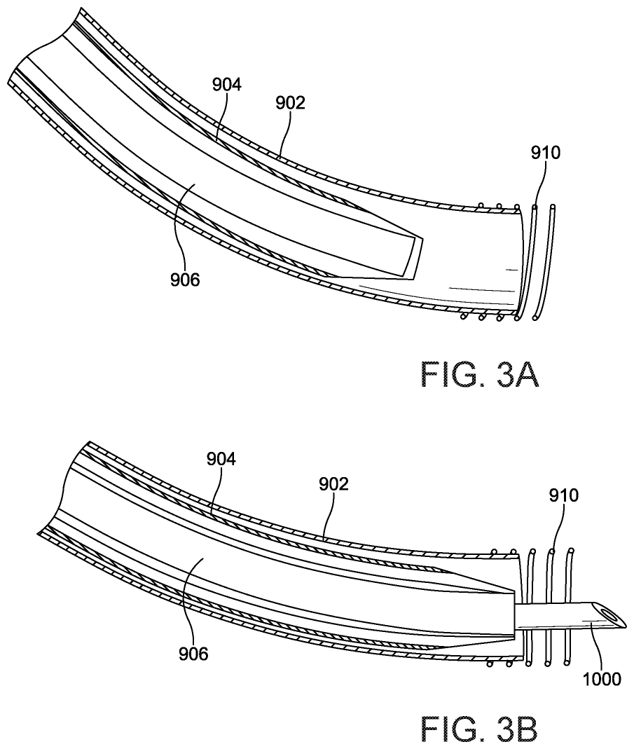

FIG. 3A illustrates the catheter 906 within the transseptal sheath 904, which are both at least partially enclosed by the access sheath 902. FIG. 3B shows that a transseptal needle 1000 may be advanced outside of the catheter 906 and the transseptal sheath 904. As such, the anchor 910 may engage a surface such as a portion of a septum of a heart and may stabilize the septum. While the anchor 910 is engaged with the surface, the needle 1000 may be advance to puncture the surface.

As an illustrative example, the anchor 910 (e.g., anchoring) coil may allow for stabilization of a fossa ovalis of a heart, preventing excessive tenting and simplifying a transseptal puncture. The access sheath 902 may be externally placed over any commercially available transseptal sheath 904. Once the transseptal sheath 904 is properly positioned in a superior vena cava of the heart and the fossa ovalis is tented with a blunt dilator, a locking mechanism (e.g., Tuohy Borst valve) on the access sheath 902 may be disengaged and the catheter 906 may be able to rotate freely about an axis of the transseptal sheath 904. The transseptal sheath 904 may be advanced until the anchor 910 at the tip is in-contact with the fossa ovalis The access sheath 902 may be rotated to advance the anchor 910 into the fossa ovalis, thereby securing the access device 900 in place. A septum of the heart may be punctured with the needle 1000 such as any commercially available transseptal needle. The transseptal sheath 904 and a dilator assembly (not shown) may be advanced into the left atrium for access. The access sheath 902 may be removed by rotating the sheath 902 counterclockwise until the anchor 910 fully disengages with the fossa ovalis.

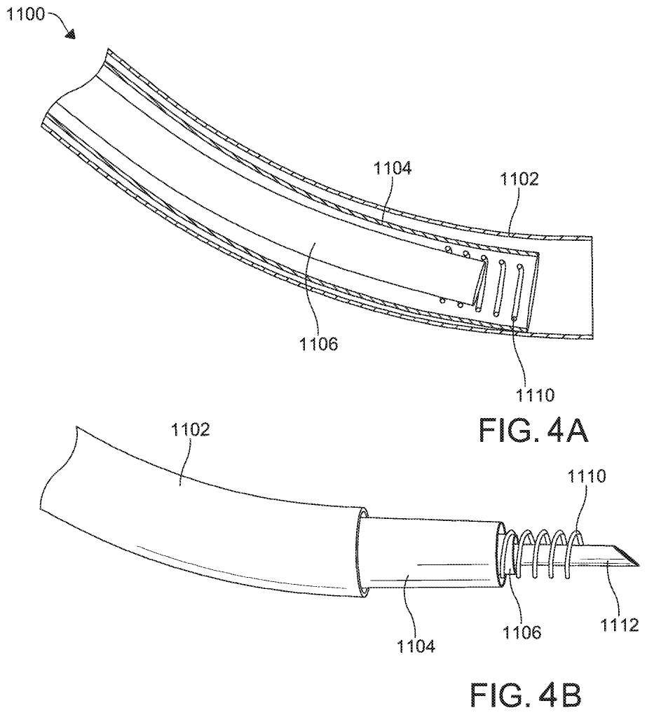

It is contemplated that the anchor 910 may be disposed in various configuration relative the other components. FIG. 4A illustrates a device 1100 comprising an outer sheath 1102 (e.g., access sheath), a transseptal sheath 1104, and a catheter 1106, for example. The components 1102, 1104, 1106 may be similar to the components 1002, 1004, 1006. As shown, however, an anchor 1110 is interposed between a portion of the catheter 1106 and a portion of the transseptal sheath 1104. The anchor 1110 extends beyond an end of the catheter 1106 and may be advanced beyond ends of the transseptal sheath 1104 and the outer sheath 1102 in order to engage a surface.

FIG. 4B shows that a transseptal needle 1112 may be advanced outside of the catheter 1106 and the transseptal sheath 1104. As such, the anchor 1110 may engage a surface such as a portion of a septum of a heart and may stabilize the septum. While the anchor 1110 is engaged with the surface, the needle 1112 may be advance to puncture the surface.

An example device may comprise a shaft comprising a hollow body, an anchor disposed adjacent an end of the shaft, wherein the anchor is configured to engage a surface to releasably secure the shaft to the surface, and a needle at least partially disposed within the shaft and is configured to be advanced toward the surface and outside of the shaft. The hollow body of the shaft may be generally tubular. The anchor may comprise a coil. The coil may be disposed to encircle at least a portion of the shaft. The coil may be configured to be advanced toward the surface in a first rotating motion to engage the surface and a second opposite rotating motion to disengage the surface. The anchor may be formed integrally with the end of the shaft or may be coupled to the end of the shaft. The anchor may be disposed along a longitudinal axis of the shaft and at least a portion of the anchor extends beyond the end of the shaft. The shaft may comprise an access sheath and the device further comprises a transseptal sheath at least partially disposed within the hollow body of the access sheath, wherein the needle comprises a transseptal needle at least partially disposed within the transseptal sheath, and wherein the transseptal needle is configured to be advanced outside of the transseptal sheath. Alternatively, the shaft may comprise a catheter and the device further comprises a transseptal sheath at least partially disposed around the hollow body of the shaft, wherein the needle comprises a transseptal needle at least partially disposed within the transseptal sheath, and wherein the transseptal needle is configured to be advanced outside of the transseptal sheath.

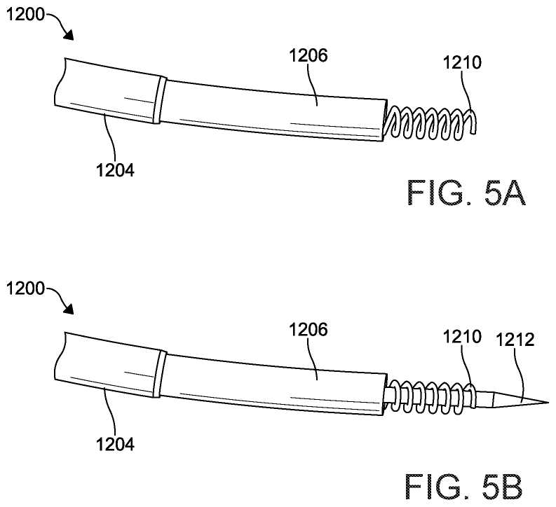

An example device 1200 is illustrated in FIG. 5A. As shown, the device 1200 may comprise a sheath 1204 such as a transseptal sheath, a catheter 1206, and an anchor 1210 such as a fixation coil. The sheath 1204 may comprise a tubular shape or a generally tubular shape. The sheath 1204 may surround at least a portion of the catheter 1206. The catheter 1206 may comprise a tubular shape or a generally tubular shape. The catheter 1206 may extend out of one side of the sheath 1204. The catheter 1206 may surround at least a portion of the anchor 1210. The anchor 1210 may comprise a corkscrew shape. The anchor 120 may extend out of one side of the catheter 1206. FIG. 5B shows that a needle 1212 may be advanced outside of the catheter 1206 and the sheath 1204. As such, the anchor 1210 may engage a surface such as a portion of a septum of a heart and may stabilize the septum. While the anchor 1210 is engaged with the surface, the needle 1212 may be advance to puncture the surface.

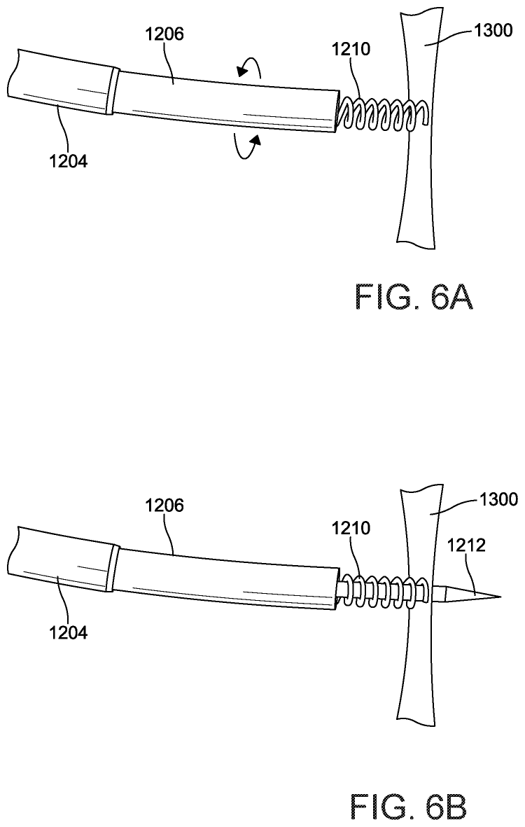

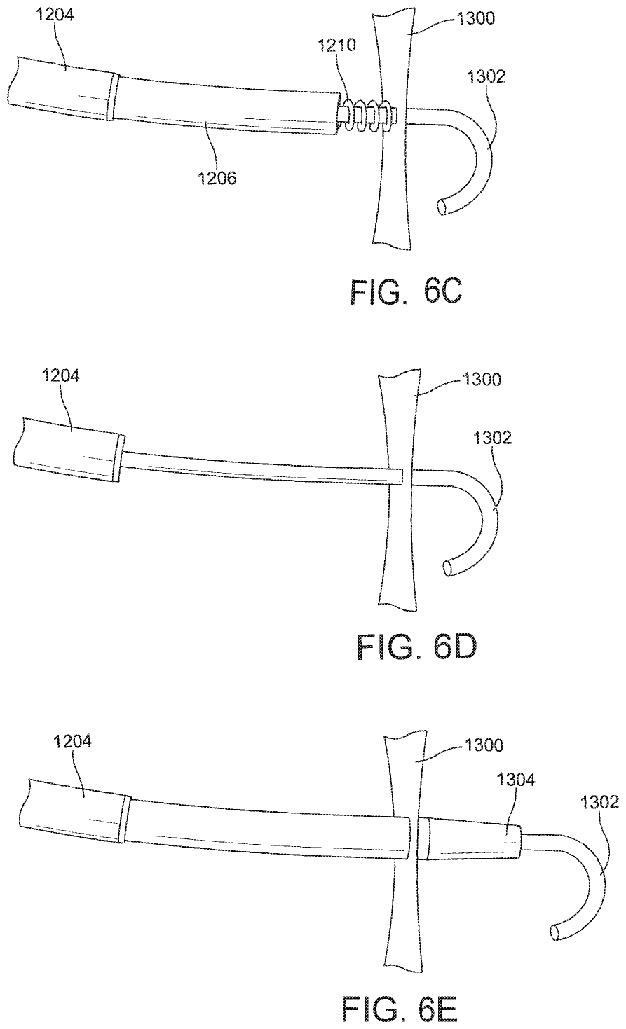

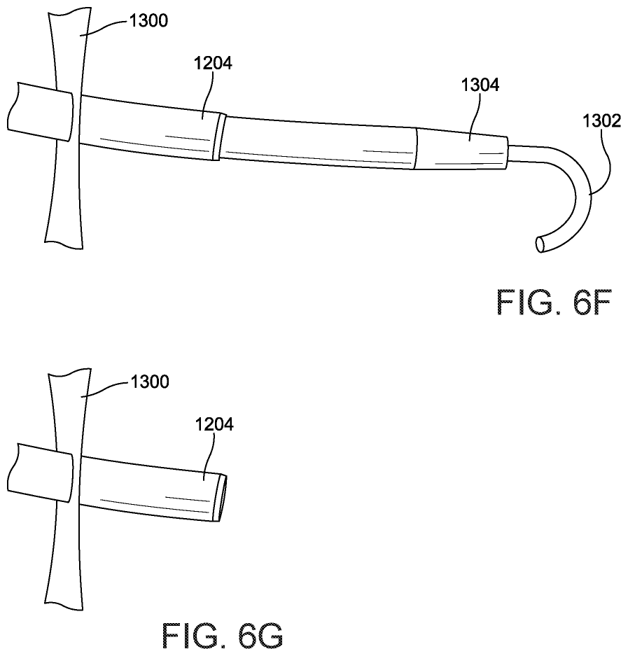

As shown, for example, in FIG. 6A, the device 1200 may be arranged such that a portion of the anchor 1210 (e.g., fixation coil) is flush with a fossa ovalis 1300 of a heart. The example system may be rotated such that the anchor 1210 is fixed to (e.g., attached to, secured to, engaged with, etc.) the surface tissue of the heart.

As shown in FIG. 6B, the needle 1210 may be advanced (e.g., pass, feed, traverse, etc.) through the example device and through the fossa ovalis 1300 to create an opening into a left atrium of the heart. The needle 1210 may be advanced through the sheath 1204, the catheter 1206, and/or the anchor 1210. The needle 1210 may then be removed.

As shown in FIG. 6C, a guidewire 1302 may be advanced through the example device 1200 and though the opening in the fossa ovalis 1300. The guidewire 1302 may be advanced through the sheath 1204, the catheter 1206, and/or the anchor 1210.

As shown in FIG. 6D, the anchor 1210 may be unfixed from (e.g., detached from, unsecured from, disengaged from, etc.) the tissue (e.g., fossa ovalis 1300). The anchor 1210 may be removed from the device 1200 via the sheath 1204. The catheter 1206 may be removed from the device 1200 via the sheath 1204. The guidewire 1302 may remain in the opening in the fossa ovalis 1300.

As shown in FIG. 6E, a transseptal dilator 1304 may be inserted over the guidewire 1302, into the opening in the fossa ovalis 1300, for example, via the sheath 1204. The transseptal dilator 1304 may be used to dilate the opening in the fossa ovalis 1300.

As shown in FIG. 6F, the sheath 1204 may be advanced into the left atrium through the dilated opening in the fossa ovalis 1300. The transseptal dilator 1304 may be removed via the sheath 1204. The guidewire 1302 may be removed via the sheath 1204. The sheath 1204 may be left in the left atrium of the heart. Further processes may be executed via the sheath 1204.

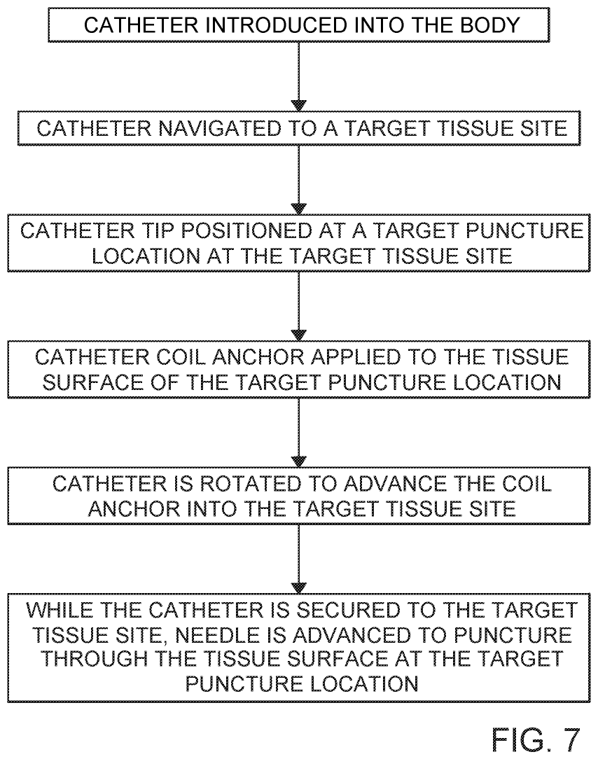

FIGS. 6A-6G illustrates example methods. Other methods may be used that relate to the heart or other tissue. Other devices, such as the devices described herein may be used and adapted for various methods. FIG. 7 illustrates an example method that may be implemented using one or more devices of the present disclosure.

The system(s), method(s), and/or device(s) disclosed herein may utilize an anchoring coil at a tip of a high-torque catheter that stabilizes a fossa ovalis of a heart, preventing excessive tenting and simplifying transseptal puncture.

The system(s), method(s), and/or device(s) disclosed herein may comprise an anchoring catheter compatible with commercially available transseptal sheaths, dilators, and transseptal needles.

The system(s), method(s), and/or device(s) disclosed herein may comprise a catheter that facilitates a secure hold to a septum of a heart so that a transseptal needle may be advanced with precise control.

The system(s), method(s), and/or device(s) disclosed herein may comprise a catheter with locking features to stabilize a transseptal sheath and prevent unwanted movement or migration during transseptal needle advancement.

The system(s), method(s), and/or device(s) disclosed herein may comprise a catheter with radiopaque markers to enhance physician visibility during a procedure.

The system(s), method(s), and/or device(s) disclosed herein may comprise a catheter that facilitates transseptal puncture of an aneurysmal septum and/or a fibrotic septum with ease.

The system(s), method(s), and/or device(s) disclosed herein may comprise a low-cost catheter that significantly reduces the difficulty and risk of transseptal punctures.

The system(s), method(s), and/or device(s) disclosed herein may comprise a low-cost catheter that provides left atrial access with minimal changes to existing procedural workflow.

The present disclosure relates to novel methods and devices for improving the positioning and functional operation of an access catheter when interfacing with various anatomical structures and/or biological surfaces. The present disclosure relates to novel methods and devices configured to assist with determining catheter orientation within the body.

The disclosure relates to methods and devices for stabilizing the distal end of a transseptal access catheter when interfacing with highly variable cardiac tissue geometries. Such stabilizing may enhance the determination of catheter position and orientation within the body, and may improve physician confidence in the positioning and operation of the access catheter via improved tactile feedback.

The present disclosure comprises an access catheter attachment device comprising an inner sheath slidably disposed within an outer sheath. In one exemplary embodiment, the inner sheath of the device is configured for the introduction of a transseptal access catheter and comprises a deployable stabilization cone disposed at one end (e.g., distal) thereof. The stabilization cone may be constrained and held in an undeployed, coaxial state by an outer sheath. The outer sheath may be configured to be withdrawn proximally along the shaft of the inner sheath to facilitate the radial deployment of the stabilization cone of the inner sheath.

In one exemplary embodiment, the stabilization cone comprises a flexible material comprising a conical geometry in its unconstrained, relaxed state. By adjusting the position of the outer sheath relative to the inner sheath, the maximum diameter of the stabilization cone at the terminal aspect of the inner sheath may be selectively modified to optimize the robustness of the access catheter attachment device to varying anatomical structures. The cone may comprise metal or nitinol spines impregnated within or disposed adjacent the cone. The inner sheath may comprise a lumen which allows any existing, commercially available transseptal puncture catheter assembly to be fitted, making the overall device compatible with a transseptal puncture (TSP) procedure.

The present disclosure comprises an attachment device that may be assembled with any existing commercially available transseptal puncture catheters. The attachment device adds a new step to a conventional transseptal puncture procedure that increases stability during the puncture step. The attachment device may comprise a cone sheath (e.g., inner sheath) and an outer sheath. The cone sheath may be configured to be disposed over a catheter. The cone sheath may comprise a tubular main body that runs the length of the catheter and terminates at and end with a flexible cone. The cone is held in a collapsed position by the outer sheath, which fits over the cone sheath.

Transseptal puncture devices, apparatuses, and methods are discussed herein. In the following description, for purposes of explanation, numerous specific details are set forth in order to provide a thorough understanding of the present invention. It will be evident, however, to one skilled in the art that the present invention may be practiced without these specific details. The present disclosure is to be considered as an exemplification of the invention, and is not intended to limit the invention to the specific embodiments illustrated by the figures or description below.

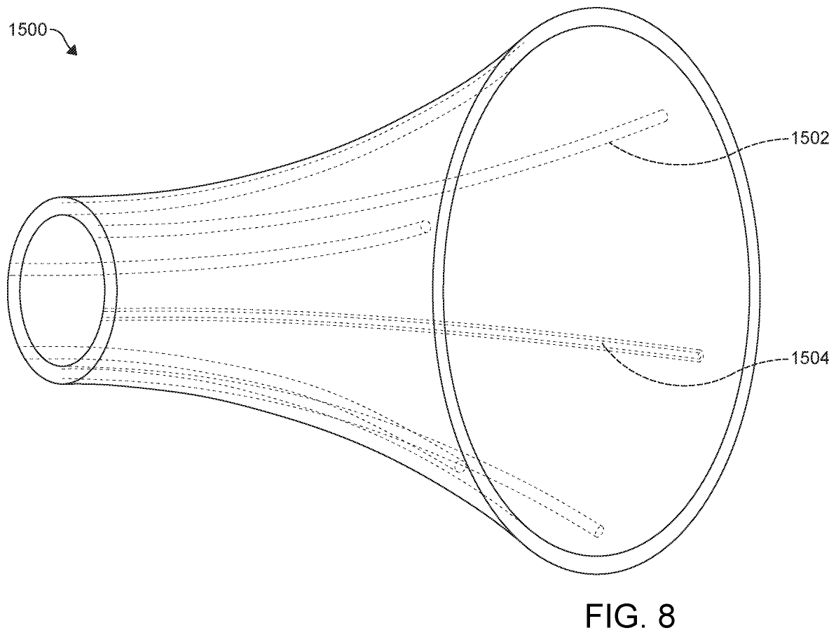

FIG. 8 illustrates a cone 1500 in accordance with the present disclosure. The cone 1500 may comprise or be formed from an elastomeric resin or any appropriately flexible material. The cone 1500 may comprise one or more lumen 1502 configured for structural supports to be received therein. In the example illustrated in FIG. 8, the cone 1500 comprises six curved metal wires that act as spines 1504 to support the flexible cone material. Additionally, these spines 1504 and/or any other part of the cone 1500 may comprise or may be formed from a radiopaque material, providing additional visual feedback to the physician should they choose to employ a technique such as fluoroscopy during the procedure.

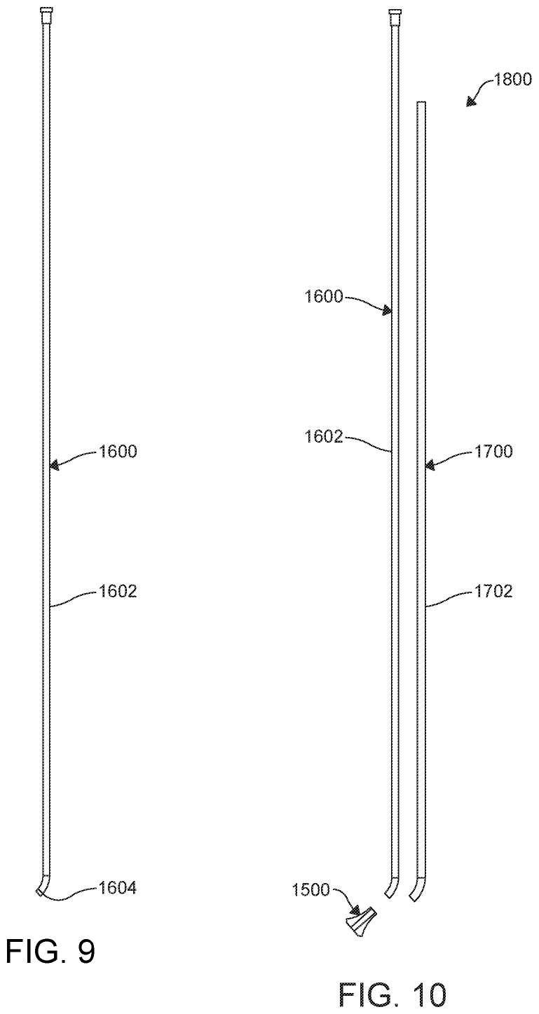

As shown in FIGS. 9-11, the cone 1500 is configured to be integrated with or coupled to the end of a cone sheath 1600 (e.g., inner sheath) with glue (e.g., UV glue) or any appropriate adhesive for use inside the body.

FIG. 9 illustrates the cone sheath 1600, which may also be referred to as an inner sheath in this disclosure. The cone sheath 1600 comprises a main body 1602 having a generally tubular shape defining a cavity that is configured to receive a conventional catheter and components of the catheter. As an example, a conventional catheter may be slid into the cone sheath 1600 into which it securely fits. The cone sheath 1600 may be sized to runs the length of the catheter. For example, available catheters exist in lengths ranging from 65 cm to 90 cm. Other lengths may be used. The cone sheath 1600 may comprise or be formed from any medical grade elastomer, such as elastomer PEBAX®, the industry standard for catheter sheaths. The cone sheath 1600 may comprise one or more markings 1604 disposed on the main body 1602 that indicate a distance an outer sheath (e.g., outer sheath 1700 (FIGS. 17-18) may be moved to deploy or resheath the cone 1500. The markings 1604 on the cone sheath 1600 may correlate to the level of radial expansion of the cone.

FIG. 10 illustrates an assembly 1800 in a disassembled state, the assembly 1800 comprising the cone 1500, the cone sheath 1600, and the outer sheath 1700. The outer sheath 1700 comprises a main body 1702. The main body 1702 has a generally tubular shape defining a cavity configured to receive the cone sheath 1600. As an illustrative example, a user may advance the outer sheath 1700 to compress/collapse the cone 1500, allowing the entire assembly to be retracted and safely removed from the patient's body. The outer sheath 1700 may comprise or be formed from the same elastomeric material, or similar, as the cone sheath 1600. The outer sheath 1700 may comprise or be formed from a different elastomeric material as the cone sheath 1600. Other materials may be used.

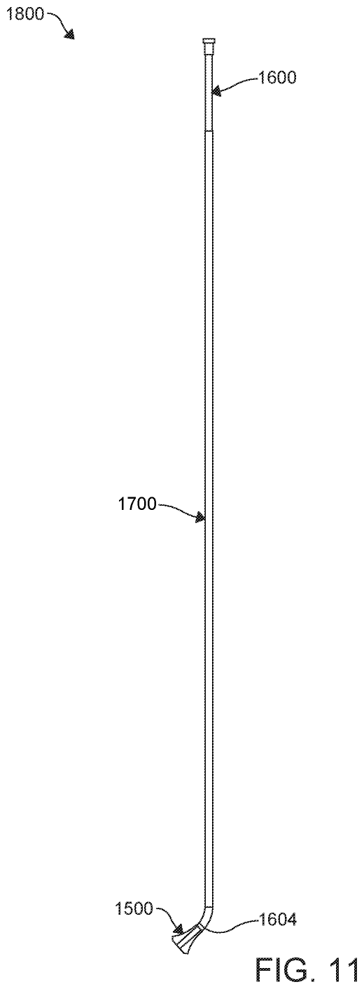

FIG. 11 illustrates the assembly 1800 in an assembled state. All components of the assembly 1800 may be formed from medical grade materials since these materials will minimize the risk for complications such as infection as this device will be inserted into the vasculature.

In operation, the cone 1500 allows for stabilization at the fossa ovalis, preventing excessive tenting and simplifying transseptal puncture. Once the transseptal sheath is properly positioned at the fossa ovalis, the outer sheath 1700 is drawn back allowing the cone 1500 to expand. Pressure is applied to the cone 1500 where it is in contact with the fossa ovalis tissue, thereby providing more stability than merely tenting with the blunt dilator. This increase in stability is facilitated at least by the fact that the cone 1500 provides 360° of contact with the fossa rather than single point contact provided by the existing assembly. This larger area of contact prevents the needle housed within the assembly from slipping in any particular direction, reducing the amount of time needed in the procedure and reducing tissue morbidity at the fossa ovalis. The fossa ovalis is then punctured with any commercially available transseptal needle and the transseptal sheath and dilator assembly can be advanced into the left atrium for access.

Clinical Impact

There are currently two device-based approaches to access a left atrium of a heart: mechanical transseptal needles, and transseptal needles outfitted with a radiofrequency (RF) electrode (RF-enabled transseptal devices). Mechanical transseptal needles, such as the BRK-1, are the most common type of device used for transseptal puncture and are reimbursable. However, mechanical transseptal needles are associated with serious complications arising from the high, uncontrollable force required to successfully puncture an atrial septum of a heart. RF-enabled transseptal devices, such as the Baylis NRG, use a blunt-tipped electrode to deliver a short and highly focused RF energy pulse to puncture a septum without the need for excessive mechanical force. Although RF-enabled transseptal devices reduce the risk of serious complications, RF-enabled transseptal devices require use of an RF generator and other additional accessories that increase procedural costs.

The sheaths and anchors disclosed herein is a cost-effective solution for simplifying left atrial access and offers significant benefits over existing devices, such as mechanical transseptal needles and RF-enabled transseptal devices, by reducing complications without negatively impacting procedural workflow.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Although the present invention has been illustrated and described herein with reference to preferred embodiments and specific examples thereof, it will be readily apparent to those of ordinary in the art that other embodiments and examples may perform similar function and/or achieve like results. All such equivalent embodiments and examples are within the spirit and scope of the present invention, are contemplated thereby, and are intended to be covered by the aforementioned claims.

What is claimed is:

1. A device comprising:
a catheter comprising a hollow body configured to enclose at least a portion of at least one of a transseptal sheath, a dilator, or a transseptal needle; and
an anchor disposed adjacent an end of the catheter, wherein the anchor is configured to engage a surface to releasably secure the catheter to the surface.

2. The device of claim 1, wherein the hollow body of the catheter is generally tubular.

3. The device of claim 1, wherein the anchor comprises a helical coil.

4. The device of claim 1, wherein the anchor comprises a helical coil configured to be advanced toward the surface in a first rotating motion to engage the surface and a second opposite rotating motion to disengage the surface.

5. The device of claim 1, wherein the anchor comprises a tip or an edge configured to pierce at least a portion of the surface.

6. The device of claim 1, wherein the surface comprises a biological surface.

7. The device of claim 1, wherein the surface comprises the fossa ovalis of a heart.

8. The device of claim 1, further comprising:
a transseptal sheath at least partially disposed within the hollow body of the catheter; and
one or more of:
a transseptal needle at least partially disposed within the transseptal sheath; or
a dilator at least partially disposed within the transseptal sheath.

9. The device of claim 8, wherein the catheter is configured for the advancement of one or more of the transseptal sheath, the dilator, or the transseptal needle along a longitudinal axis of the catheter.

10. The device of claim 1, wherein the catheter is slidably disposed along a shaft of one or more of a transseptal sheath, a dilator, or a transseptal needle.

11. The device of claim 1, wherein the catheter is rotationally disposed along a shaft of one or more of a transseptal sheath, a dilator, or a transseptal needle.

12. The device of claim 1, wherein the catheter is slidably and rotationally disposed along a shaft of one or more of a transseptal sheath, a dilator, or a transseptal needle.

13. The device of claim 1, further comprising one or more radiopaque markers disposed on or adjacent the hollow body of the catheter.

14. The device of claim 1, further comprising a locking element configured to secure the position of the catheter relative to at least one of a transseptal sheath, a dilator, or a transseptal needle.

15. A device comprising:
a shaft comprising a hollow body;
an anchor disposed adjacent an end of the shaft, wherein the anchor is configured to engage a surface to releasably secure the shaft to the surface; and
a needle at least partially disposed within the shaft and configured to be advanced toward the surface and beyond an end of the shaft.

16. The device of claim 15, wherein the hollow body is generally tubular.

17. The device of claim 15, wherein the anchor comprises a helical coil.

18. The device of claim 17, wherein the helical coil is disposed to encircle at least a portion of the shaft.

19. The device of claim 15, wherein the anchor comprises a helical coil configured to be advanced toward the surface in a first rotating motion to engage the surface and a second opposite rotating motion to disengage the surface.

20. The device of claim 15, wherein the shaft is slidably disposed along a length of the needle.

21. The device of claim 15, wherein the shaft is rotationally disposed along a length of the needle.

22. The device of claim 15, wherein the shaft is slidably and rotationally disposed along a length of the needle.

23. The device of claim 15, further comprising one or more radiopaque markers disposed on or adjacent the hollow body of the shaft.

24. The device of claim 15, further comprising a locking element configured to secure the position of the shaft relative to the needle.

25. The device of claim 15, wherein the anchor comprises a tip or an edge configured to pierce at least a portion of the surface.

26. The device of claim 15, wherein the anchor is formed integrally with the end of the shaft.

27. The device of claim 15, wherein the anchor is coupled to the end of the shaft.

28. The device of claim 15, wherein the anchor is disposed along a longitudinal axis of the shaft and at least a portion of the anchor extends beyond the end of the shaft.

29. The device of claim 15, wherein the surface comprises a biological surface.

30. The device of claim 15, wherein the surface comprises the fossa ovalis of a heart.

31. The device of claim 15, wherein the shaft comprises a transseptal sheath and wherein the needle comprises a transseptal needle at least partially disposed within the transseptal sheath.

32. The device of claim 31, further comprising a dilator at least partially disposed within the transseptal sheath.

33. The device of claim 32, wherein the dilator is configured to be advanced outside of the transseptal sheath.

34. The device of claim 31, wherein the transseptal sheath is configured to be slidably disposed along an axis of the transseptal needle.

35. The device of claim 31, wherein the transseptal sheath is configured to be rotationally disposed along an axis of the transseptal needle.

36. The device of claim 31, wherein the transseptal sheath is configured to be slidably and rotationally disposed along an axis of the transseptal needle.

37. The device of claim 31, wherein the transseptal sheath is configured for the advancement of the transseptal needle.

38. The device of claim 15, wherein the shaft comprises a catheter and the device further comprises a transseptal sheath at least partially disposed within the hollow body of the shaft, wherein the needle comprises a transseptal needle at least partially disposed within the transseptal sheath, and wherein one or more of the transseptal sheath or transseptal needle is configured to be advanced outside of the catheter.

39. The device of claim 15, wherein the shaft comprises a catheter and the device further comprises a transseptal sheath at least partially disposed around the hollow body of the catheter, wherein the needle comprises a transseptal needle at least partially disposed within the hollow body of the catheter, and wherein the transseptal needle is configured to be advanced outside of the catheter.

40. The device of claim 15, further comprising a dilator at least partially disposed within the hollow body of the shaft.

41. The device of claim 40, wherein the dilator is configured to be advanced outside of the hollow body of the shaft.

42. A method comprising:
disposing a device adjacent a biological surface, wherein the device comprises at least a shaft having a hollow body and a needle disposed at least partially within the hollow body of the shaft;
causing the device to engage the surface to releasably secure at least a portion of the device to the surface; and
causing the needle to be advanced toward the surface and outside of the hollow body of the shaft, while the at least a portion of the device is secured to the surface.

43. The method of claim 42, wherein the device comprises an anchor disposed adjacent an end of the shaft, wherein the anchor is configured to engage the surface to releasably secure the shaft to the surface.

44. The method of claim 42, wherein the causing the device to engage the surface comprises using a helical coil.

45. The method of claim 42, wherein the causing the device to engage the surface comprises using a helical coil configured to be advanced toward the surface in a first rotating motion to engage the surface and a second opposite rotating motion to disengage the surface.

46. The method of claim 42, wherein the causing the device to engage the surface comprises using a tip or an edge of an anchor configured to pierce at least a portion of the surface.

47. The method of claim 42, wherein the surface comprises the fossa ovalis of a heart.

48. The method of claim 47, further comprising causing the needle to be advance through at least a portion of an interatrial septum of the heart, while the at least a portion of the device is secured to the fossa ovalis.

49. The method of claim 48, further comprising causing the needle to be advance through at least a portion of the fossa ovalis of the heart, while the at least a portion of the device is secured to the fossa ovalis.

50. The method of claim 49, further comprising causing the needle to be advance into a left atrium of the heart, while the at least a portion of the device is secured to the fossa ovalis.

51. An assembly comprising
a cone stabilizer having an expandable and contractible main body having a conical shape;
a cone sheath having a tubular main body configured to receive a catheter, wherein the cone stabilizer is disposed at an end of the cone sheath; and
an outer sheath having a tubular main body configured to slidably receive the cone sheath therein, wherein a select movement of the cone sheath relative to the outer sheath allows the main body of the cone stabilizer to expand or contract.

52. The assembly of claim 51, wherein an axial movement of the outer sheath relative to the cone sheath allows the main body of the cone stabilizer to expand or contract in a radial direction.

53. The assembly of claim 51, wherein the outer sheath is configured to be disposed around the cone stabilizer to constrict a radial expansion of the main body of the cone.

54. The assembly of claim 51, wherein the main body of the cone comprises one or more support spines.
